(19) United States
(12) Patent Application Publication
(10) Pub. No.: US 2006/0210497 A1
(43) Pub. Date: Sep. 21, 2006

(54) Novel resorcinol derivatives

(75) Inventors: Bijan Harichian, Warren, NJ (US); Jose Guillermo Rosa, Edgewater, NJ (US); John Steven Bajor, Cheshire, CT (US)

Correspondence Address: UNILEVER INTELLECTUAL PROPERTY GROUP, 700 SYLVAN AVENUE,, BLDG C2 SOUTH, ENGLEWOOD CLIFFS, NJ, 07632-3100, US

(73) Assignee: Unilever Home & Personal Care USA, Division Of Conopco, Inc.

(21) Appl. No.: 11/083,626

(22) Filed: Mar. 18, 2005

(51) Int. Cl. C07C 39/12; A61K 8/34

(52) U.S. Cl. 424/62; 568/731

(57) ABSTRACT

New 4-substituted resorcinol derivatives of general formula I and/or B and process for synthesizing same, cosmetic compositions and methods of using same, particularly for skin lightening and/or reducing the appearance of oily or greasy skin: 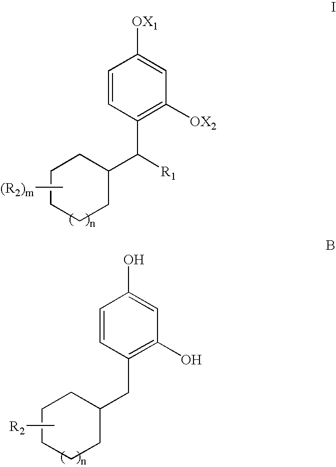 Wherein
X₁ and/or X₂ represents hydrogen (H), linear or branched, saturated or unsaturated C₁-C₁₂ alkyl, alkenyl, or acyl groups. Preferably, X₁ and/or X₂ represents hydrogen (H); linear or branched, saturated or unsaturated C₁-C₁₂ alkyl or acyl groups;
R₁ and/or R₂ represents hydrogen (H), linear or branched, cyclic or acyclic, saturated or unsaturated C₁-C₁₂ alkyl, alkenyl, cycloalkyl, or cycloalkenyl group; n represents 0, 1. When n=0, the ring is a cyclopentyl with or without one heteroatom from O, N or S and/or with or without one double bond. When n=1, the ring is a cyclohexyl with or without one heteroatom from O, N or S and/or with or without one double bond; and m represents an integer between 1 and 6.

### FIELD OF THE INVENTION

The invention relates to new 4-substituted resorcinol derivatives, cosmetic compositions containing same, and cosmetic methods of using and making same. More specifically, the present invention relates to new 4-substituted resorcinol derivatives as skin cosmetic actives, cosmetic compositions and methods of using same for skin lightening, reducing the appearance of oily or greasy skin and/or other cosmetic skin benefits.

### BACKGROUND OF THE INVENTION

Many people are concerned with the degree of pigmentation of their skin. For example, people with age spots or freckles may wish such pigmented spots to be less pronounced. Others may wish to reduce the skin darkening caused by exposure to sunlight or to lighten their natural skin color. To meet this need, many attempts have been made to develop products that reduce the pigment production in the melanocytes. However, the substances identified thus far tend to have either low efficacy or undesirable side effects, such as, for example, toxicity or skin irritation. Therefore, there is a continuing need for new skin lightening agents, with improved overall effectiveness.

Sebum is skin oil which is produced by sebocytes (cells of the sebaceous glands in the skin) and is then secreted to the skin surface. Sebum is made up of a number of components, with about 57% being triglycerides, or fatty acids. About 12% of sebum is squalene, about 3% of sebum are sterol esters, about 25% wax esters, and about 1 to about 2% cholesterol. A frequent and undesirable skin condition is “oily skin,” the condition which results from the excessive amount of sebum on the skin. Oily skin is associated with a shiny, undesirable appearance and a disagreeable tactile sensation and affects various age groups. Excessive sebum production is cosmetically undesirable and has been associated with the skin condition acne. Therefore, cosmetic products and methods that provide sebum control or reduce the appearance of oily or greasy skin are highly desirable.

Resorcinol derivatives are generally known compounds and can be readily obtained, for example, by a method wherein a saturated carboxylic acid and resorcinol are condensed in the presence of zinc chloride and the resultant condensate is reduced with zinc amalgam/hydrochloric acid (Lille. J. Bitter, L A. Peiner. V, Tr. Nauch-Issled. Inst. slantsev 1969, No. 18, 127), or by a method wherein resorcinol and a corresponding alkyl alcohol are reacted in the presence of an alumina catalyst at a high temperature of from 200 to 400° C. (British Patent No. 1,581,428).

Resorcinol derivatives have cosmetic skin and hair benefits. Certain resorcinol derivatives, particularly 4-substituted resorcinol derivatives, are useful in cosmetic compositions for skin lightening benefits. Resorcinol derivatives are described in many publications, including U.S. Pat. No. 4,959,393; Hu et al., U.S. Pat. No. 6,132,740; Bradley, et al., U.S. Pat. No. 6,504,037; and Japanese published patent applications JP 2001-010925 and JP2000-327557. Skin lightening compounds that may be derived from coumarin are disclosed in U.S. Patent Publication No. 2004/0042983. Some of these compounds can be difficult to formulate and/or irritating to the skin.

Applicants have now discovered new compounds that deliver skin cosmetic benefits, particularly skin lightening benefits.

In another aspect, the present invention is based on the discovery that the 4-substituted resorcinol derivatives reduce the appearance of oily or greasy skin.

The general chemical formulas and structures of these compounds are discussed in more detail herein below. In particular, the novel 4-substituted resorcinol derivatives, have been found to be cosmetically effective to the skin.

### SUMMARY OF THE INVENTION

Applicants have now discovered new 4-substituted resorcinol derivatives that have skin cosmetic activity, including skin lightening and/or sebum suppressive activity. Accordingly, the present invention provides novel compounds of general formula I, cosmetic compositions and methods comprising same, particularly for skin lightening and/or reducing the appearance of oily or greasy skin, as well as a process for producing the inventive compounds: 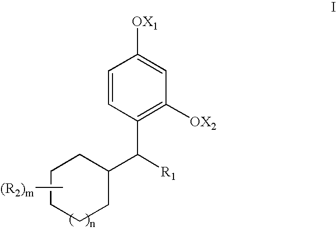
Wherein
X₁ and/or X₂ represents hydrogen (H), linear or branched, saturated or unsaturated C₁-C₁₂ alkyl, alkenyl, acyl groups. Preferably, X₁ and/or X₂ represents hydrogen (H); linear or branched, saturated or unsaturated C₁-C₁₂ alkyl or acyl groups. More preferably, X₁ and/or X₂ represents hydrogen.
R₁ and/or R₂ represents hydrogen (H), linear or branched, cyclic or acyclic, saturated or unsaturated C₁-C₁₂ alkyl, alkenyl, cycloalkyl, or cycloalkenyl group.
Preferably, R₁, and/or R₂ represents hydrogen (H) or C₁ alkyl group (i.e, methyl group). More preferably, R₁ and/or R₂ represents hydrogen.
n represents 0, 1. When n=0, the ring is a cyclopentyl with or without one heteroatom, such as from O, N or S, and/or with or without one double bond. When n=1, the ring is a cyclohexyl with or without one heteroatom, such as from O, N or S, and/or with or without one double bond.
m=1, 2, 3, 4, 5, 6 (i.e, an integer between 1 and 6).
Where m is 2, R₂ may or may not be fused.
   Preferred compounds include:
n=0, m=1, R₂=H, alkyl, and/or alkenyl; anywhere in the ring;
n=1, m=1, R₂=H, alkyl, and/or alkenyl; anywhere in the ring;
n=0, m=2-5, R₂=H, alkyl, and/or alkenyl; any substitution pattern anywhere in the ring;
n=1, m=2-6, R₂=H, alkyl and/or alkenyl; any substitution pattern anywhere in the ring.

Where R₁, X₁ and X₂ are each H, and m is 1, the compound is represented by formula B:

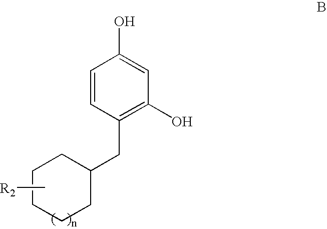

Cosmetic compositions according to the present invention include: a.about 0.0001 wt. % to about 50 wt. %, preferably about 0.01 wt. % to about 5 wt. %, of a 4-substituted resorcinol derivative of the general formula I, preferably of formula B; and
b. a cosmetically acceptable vehicle.

The inventive compositions may further include an organic sunscreen selected from the group consisting of Benzophenone-1, Benzophenone-2, Benzophenone-3, Benzophenone-4, Benzophenone-8, DEA, Methoxycinnamate, Ethyl dihydroxypropyl-PABA, Glyceryl PABA, Homosalate, Methyl anthranilate, Octocrylene, Octyl dimethyl PABA, Octyl methoxycinnamate (PARSOL MCX), Octyl salicylate, PABA, 2-Phenylbenzimidazole-5-sulphonic acid, TEA salicylate, 3-(4-methylbenzylidene)-camphor, Benzophenone-6, Benzophenone-12, 4-Isopropyl dibenzoyl methane, Butyl methoxy dibenzoyl methane (PARSOL 1789), Etocrylene, and mixtures thereof.

Additionally, the inventive cosmetic compositions may further include a skin benefit agent selected from the group consisting of alpha-hydroxy acids and esters, beta-hydroxy acids and esters, polyhydroxy acids and esters, kojic acid and esters, ferulic acid and ferulate derivatives, vanillic acid and esters, dioic acids and esters, retinol, retinal, retinyl esters, creatine, hydroquinone, t-butyl hydroquinone, mulberry extract, licorice extract, resorcinol derivatives, and mixtures thereof.

The inventive compounds and compositions may be applied to the skin as part of inventive cosmetic method for skin lightening and/or reducing the appearance of oily or greasy skin, preferably, for skin lightening.

In another aspect, the present invention relates to a process for synthesizing the novel resorcinol compounds of general formula I, having one of the following two general reaction schemes: 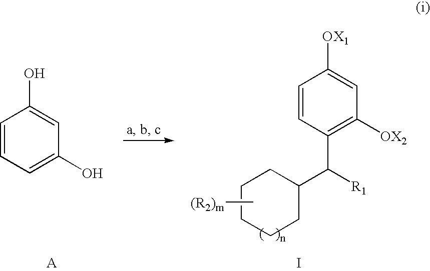
Wherein a, b, c represent reaction steps, reagents and conditions:
(a) α-alkylbenzyl alcohol derivative (or substituted α-alkylbenzyl alcohol derivative, e.g. having m number of R₂ groups), acid catalyst;
(b) hydrogen, catalyst (e.g. 5% Pd/C), solvent (e.g. acidic conditions);
(c) acylation (e.g., acetic anhydride, triethylamine) or alkylation (e.g. alkyl halide, base); or 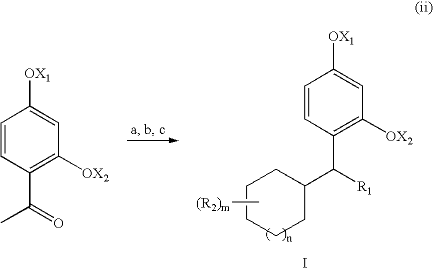
   Reagents and conditions:
(a) cycloalkylmagnesium halide derivative (or substituted cycloalkylmagnesium halide derivative, e.g. having m number of R₂ groups), organic solvent;
(b) acidic conditions (e.g. acetic anhydride);
(c) hydrogen, catalyst (e.g. 5% Pd/C), solvent (e.g. acidic conditions).

### DETAILED DESCRIPTION OF THE INVENTION

The invention is concerned with new 4-substituted resorcinol derivatives for cosmetic skin benefit, including lightening skin color and/or reducing the appearance of oily or greasy skin, cosmetic compositions and methods employing same, and a process for producing same.

As used herein, the term “cosmetic composition” is intended to describe compositions for topical application to human skin, including leave-on and wash-off products.

The term “skin” as used herein includes the skin on the face, neck, chest, back, arms, axillae, hands, legs, and scalp.

Except in the examples, or where otherwise explicitly indicated, all numbers in this description indicating amounts of material or conditions of reaction, physical properties of materials and/or use are to be understood as modified by the word “about”. All amounts are by weight of the composition, unless otherwise specified.

For the avoidance of doubt, the term “comprising” means including, made up of, composed of, consisting and/or consisting essentially of. Furthermore, in the ordinary meaning of “comprising,” the term is defined as not being exhaustive of the steps, components, ingredients, or features to which it refers.

The term “reducing the appearance of oily or greasy skin” is meant herein to refer to any detectable reduction in skin sebum, e.g., a reduction visible to the naked eye, that occurs after contacting the skin of an individual with a composition comprising a sebum suppressive active.

Novel 4-Substituted Resorcinol Derivatives

The present invention is based on a new resorcinol derivative of the general formula I. Accordingly, the present invention provides novel compounds of general formula I, cosmetic compositions and methods comprising same, particularly for skin lightening and/or reducing the appearance of oily or greasy skin, as well as a process for producing the inventive compounds: 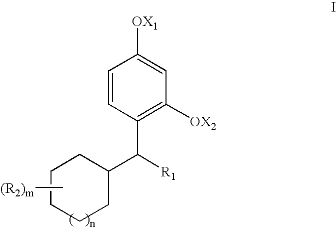
Wherein
X₁ and/or X₂ represents hydrogen (H), linear or branched, saturated or unsaturated C₁-C₁₂ alkyl, alkenyl, or acyl groups. Preferably, X₁ and/or X₂ represents hydrogen (H); linear or branched, saturated or unsaturated C₁-C₁₂ alkyl or acyl groups.
R₁ and/or R₂ represents hydrogen (H), linear or branched, cyclic or acyclic, saturated or unsaturated C₁-C₁₂ alkyl, alkenyl, cycloalkyl, or cycloalkenyl group. Preferably, R₁ and/or R₂ represents hydrogen (H) or C₁ alkyl group (i.e, methyl group). More preferably, R₁ and/or R₂ represents hydrogen.
n represents 0, 1. When n=0, the ring is a cyclopentyl with or without one heteroatom, such as from O, N or S, and/or with or without one double bond. When n=1, the ring is a cyclohexyl with or without one heteroatom, such as from O, N or S, and/or with or without one double bond.
m=1, 2, 3, 4, 5, 6 (i.e, an integer between 1 and 6).
Where m is 2, R₂ may or may not be fused.
   Preferred compounds include:
n=0, m=1, R₂=H, C₁-C₁₂ alkyl or alkenyl; anywhere in the ring.
n=1, m=1, R₂=H, C₁-C₁₂ alkyl or alkenyl; anywhere in the ring.
n=0, m=2-5, R₂=H, C₁-C₁₂ alkyl and/or alkenyl; any substitution pattern anywhere in the ring.
n=1, m=2-6, R₂=H, C₁-C₁₂ alkyl and/or alkenyl; any substitution pattern anywhere in the ring.

Examples of more specific embodiments of the 4-substituted cycloalkyl-methyl resorcinols include compounds of general formula B: 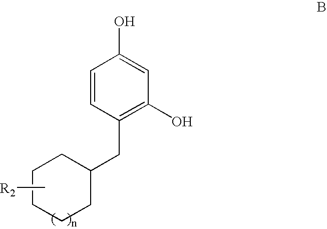
Wherein
R₂=hydrogen (H), linear or branched, cyclic or acyclic, saturated or unsaturated C₁-C₁₂ alkyl, alkenyl, cycloalkyl, or cycloalkenyl group. Preferably, R₂ represents hydrogen (H) or a C₁ alkyl group (i.e, methyl group). More preferably, R₂ represents hydrogen.
n represents 0, 1. When n=0, the ring is a cyclopentyl with or without one heteroatom from O, N or S and/or with or without one double bond. When n=1, the ring is a cyclohexyl with or without one heteroatom from O, N or S and/or with or without one double bond.

Preferred compounds are 4-cyclopentyl methyl resorcinol, 4-cyclohexyl methyl resorcinol.

Cosmetic compositions according to the present invention include:
a. about 0.0001 wt. % to about 50 wt. % of a 4-substituted resorcinol derivative of the general formula I, or preferably formula B; and
b. a cosmetically acceptable vehicle.
   The amount of the resorcinol derivative is preferably in the range of about 0.00001% to about 10%, more preferably about 0.001 to 7%, most preferably from 0.01% to about 5%, of the total amount of a cosmetic composition.

The inventive compounds and compositions may be applied to the skin as part of inventive cosmetic method for skin lightening, and/or reducing the appearance of oily or greasy skin, and/or other cosmetic skin benefits.

Without wishing to be bound by theory, Applicants believe the novel molecular structure to possess stable bonds, to be less susceptable to oxidation and discoloration, and to be more stable than other 4-substituted resorcinol derivative structures. It is also believed that the unique structure of the molecules of the present invention, particularly the methylene group, critically leads to unique and advantageous functionality. For example, the methylene group serves as a spacer between the resorcinol moiety (having flat stereo-chemistry) and the derivative moiety, providing a center of free rotation for the derivative moiety around the methylene group, which is believed, among other effects, to lead to different enzyme binding properties.

Synthetic Process for Novel Resorcinol Derivatives

The present invention includes to a process for synthesizing the novel resorcinol compounds of general formula I, having one of the following two general reaction schemes: 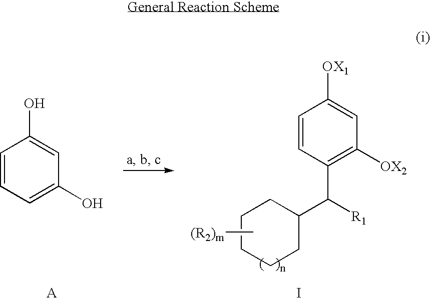
Wherein a, b, c represent reaction steps, reagents and conditions:
(a) alpha-alkylbenzyl alcohol derivative (or substituted alpha-alkylbenzyl alcohol derivative, e.g. having m number of R₂ groups), acid catalyst;
(b) hydrogen, catalyst (e.g. 5% Pd/C), solvent (e.g. acidic conditions);
(c) acylation (e.g., acetic anhydride, triethylamine) or alkylation (e.g. alkyl halide, base). 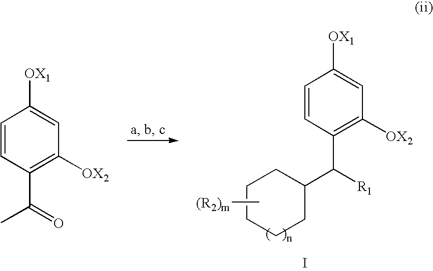
   Reagents and conditions:
(a) cycloalkylmagnesium halide derivative (or substituted cycloalkylmagnesium halide derivative, e.g. having m number of R₂ groups), organic solvent;
(b) acidic conditions (e.g. acetic anhydride);
(c) hydrogen, catalyst (e.g. 5% Pd/C), solvent (e.g. acidic conditions).

### Skin Benefit Agents

Preferred cosmetic compositions are those suitable for the application to human skin, which optionally, but preferably, include a skin benefit agent.

Suitable skin benefit agents include anti-aging, wrinkle-reducing, skin whitening, anti-acne, and sebum reduction agents. Examples of these include alpha-hydroxy acids and esters, beta-hydroxy acids and esters, polyhydroxy acids and esters, kojic acid and esters, ferulic acid and ferulate derivatives, vanillic acid and esters, dioic acids (such as sebacic and azoleic acids) and esters, retinol, retinal, retinyl esters, creatine, hydroquinone, t-butyl hydroquinone, niacinamide, mulberry extract, licorice extract, and resorcinol derivatives other than the 4-substituted resorcinol derivatives discussed hereinabove.

### Cosmetically Acceptable Carrier

The skin benefit agent together with the organic sunscreen compound and resorcinol derivative of the invention is usually used along with a cosmetic base. Suitable cosmetic carriers are well known to one skilled in the art. The cosmetic bases may be any bases which are ordinarily used for skin benefit agents and are not thus critical. Specific preparations of the cosmetics to which the skin benefit agents of the invention are applicable include creams, ointments, emulsions, lotions, oils, packs and nonwoven wipes. Cream bases are, for example, beeswax, cetyl alcohol, stearic acid, glycerine, propylene glycol, propylene glycol monostearate, polyoxyethylene cetyl ether and the like. Lotion bases include, for example, oleyl alcohol, ethanol, propylene glycol, glycerine, lauryl ether, sorbitan monolaurate and the like.

The cosmetically acceptable vehicle may act as a dilutant, dispersant or carrier for the skin benefit ingredients in the composition, so as to facilitate their distribution when the composition is applied to the skin.

The vehicle may be aqueous, anhydrous or an emulsion. Preferably, the compositions are aqueous or an emulsion, especially water-in-oil or oil-in-water emulsion, preferentially oil in water emulsion. Water when present will be in amounts which may range from 5 to 99%, preferably from 20 to 70%, optimally between 40 and 70% by weight.

Besides water, relatively volatile solvents may also serve as carriers within compositions of the present invention. Most preferred are monohydric C₁-C₃ alkanols. These include ethyl alcohol, methyl alcohol and isopropyl alcohol. The amount of monohydric alkanol may range from 1 to 70%, preferably from 10 to 50%, optimally between 15 to 40% by weight.

Emollient materials may also serve as cosmetically acceptable carriers. These may be in the form of silicone oils and synthetic esters. Amounts of the emollients may range anywhere from 0.1 to 50%, preferably between 1 and 20% by weight.

Silicone oils may be divided into the volatile and non-volatile variety. The term “volatile” as used herein refers to those materials which have a measurable vapor pressure at ambient temperature. Volatile silicone oils are preferably chosen from cyclic or linear polydimethylsiloxanes containing from 3 to 9, preferably from 4 to 5, silicon atoms. Linear volatile silicone materials generally have viscosities less than about 5 centistokes at 25° C. while cyclic materials typically have viscosities of less than about 10 centistokes. Nonvolatile silicone oils useful as an emollient material include polyalkyl siloxanes, polyalkylaryl siloxanes and polyether siloxane copolymers. The essentially non-volatile polyalkyl siloxanes useful herein include, for example, polydimethyl siloxanes with viscosities of from about 5 to about 25 million centistokes at 25° C. Among the preferred non-volatile emollients useful in the present compositions are the polydimethyl siloxanes having viscosities from about 10 to about 400 centistokes at 25° C.

Among the ester emollients are:
(1) Alkenyl or alkyl esters of fatty acids having 10 to 20 carbon atoms. Examples thereof include isoarachidyl neopentanoate, isononyl isonanonoate, oleyl myristate, oleyl stearate, and oleyl oleate.
(2) Ether-esters such as fatty acid esters of ethoxylated fatty alcohols.
(3) Polyhydric alcohol esters. Ethylene glycol mono and di-fatty acid esters, diethylene glycol mono- and di-fafty acid esters, polyethyl-ene glycol (200-6000) mono- and di-fatty acid esters, propylene glycol mono- and di-fatty acid esters, polypropylene glycol 2000 monooleate, polypropylene glycol 2000 monostearate, ethoxylated propylene glycol monostearate, glyceryl mono- and di-fatty acid esters, polyglycerol poly-fatty esters, ethoxylated glyceryl monostearate, 1,3-butylene glycol monostearate, 1,3-butylene glycol distearate, polyoxyethylene polyol fatty acid ester, sorbitan fatty acid esters, and polyoxyethylene sorbitan fatty acid esters are satisfactory polyhydric alcohol esters.
(4) Wax esters such as beeswax, spermaceti, myristyl myristate, stearyl stearate and arachidyl behenate.
(5) Sterol esters, of which cholesterol fatty acid esters are examples.

Fatty acids having from 10 to 30 carbon atoms may also be included as cosmetically acceptable carriers for compositions of this invention. Illustrative of this category are pelargonic, lauric, myristic, palmitic, stearic, isostearic, hydroxystearic, oleic, linoleic, ricinoleic, arachidic, behenic and erucic acids.

Humectants of the polyhydric alcohol-type may also be employed as cosmetically acceptable carriers in compositions of this invention. The humectant aids in increasing the effectiveness of the emollient, reduces skin dryness and improves skin feel. Typical polyhydric alcohols include glycerol, polyalkylene glycols and more preferably alkylene polyols and their derivatives, including propylene glycol, dipropylene glycol, polypropylene glycol, polyethylene glycol and derivatives thereof, sorbitol, hydroxypropyl sorbitol, hexylene glycol, 1,3-butylene glycol, 1,2,6-hexanetriol, ethoxylated glycerol, propoxylated glycerol and mixtures thereof. The amount of humectant may range anywhere from 0.5 to 30%, preferably between 1 and 15% by weight of the composition.

Thickeners may also be utilized as part of the cosmetically acceptable carrier of compositions according to the present invention. Typical thickeners include crosslinked acrylates (e.g. Carbopol 982), hydrophobically-modified acrylates (e.g. Carbopol 1382), cellulosic derivatives and natural gums. Among useful cellulosic derivatives are sodium carboxymethylcellulose, hydroxypropyl methylcellulose, hydroxypropyl cellulose, hydroxyethyl cellulose, ethyl cellulose and hydroxymethyl cellulose. Natural gums suitable for the present invention include guar, xanthan, sclerotium, carrageenan, pectin and combinations of these gums. Amounts of the thickener may range from 0.0001 to 5%, usually from 0.001 to 1%, optimally from 0.01 to 0.5% by weight.

Collectively the water, solvents, silicones, esters, fatty acids, humectants and/or thickeners will constitute the cosmetically acceptable carrier in amounts from 1 to 99.9%, preferably from 80 to 99% by weight.

An oil or oily material may be present, together with an emulsifier to provide either a water-in-oil emulsion or an oil-in-water emulsion, depending largely on the average hydrophilic-lipophilic balance (HLB) of the emulsifier employed.

Surfactants may also be present in cosmetic compositions of the present invention. For leave-on products, total concentration of the surfactant will range from 0.1 to 40%, preferably from 1 to 20%, optimally from 1 to 5% by weight of the composition. For wash-off products, such as cleansers and soap, total concentration of surfactant will range at about 1 to about 90%. The surfactant may be selected from the group consisting of anionic, nonionic, cationic and amphoteric actives. Particularly preferred nonionic surfactants are those with a C₁₀-C₂₀ fatty alcohol or acid hydrophobe condensed with from 2 to 100 moles of ethylene oxide or propylene oxide per mole of hydrophobe; C₂-C₁₀ alkyl phenols condensed with from 2 to 20 moles of alkylene oxide; mono- and di-fatty acid esters of ethylene glycol; fatty acid monoglyceride; sorbitan, mono- and di-C₈-C₂₀ fatty acids; block copolymers (ethylene oxide/propylene oxide); and polyoxyethylene sorbitan as well as combinations thereof. Alkyl polyglycosides and saccharide fatty amides (e.g. methyl gluconamides) are also suitable nonionic surfactants.

Preferred anionic surfactants include soap, alkyl ether sulfate and sulfonates, alkyl sulfates and sulfonates, alkylbenzene sulfonates, alkyl and dialkyl sulfosuccinates, C₈-C₂₀ acyl isethionates, acyl glutamates, C₈-C₂₀ alkyl ether phosphates and combinations thereof.

The inventive cosmetic compositions optionally contain a lathering surfactant. By a “lathering surfactant” is meant a surfactant which, when combined with water and mechanically agitated, generates a foam or lather. Preferably, the lathering surfactant should be mild, meaning that it must provide sufficient cleansing or detergent benefits but not overly dry the skin, and yet meet the lathering criteria described above. The cosmetic compositions of the present invention may contain a lathering surfactant in a concentration of about 0.01% to about 50%.

### Optional Components

In the cosmetic compositions of the invention, there may be added various other plasticizers, elastomers, calamine, pigments, antioxidants, chelating agents, and perfumes, as well as organic sunscreens and sunscreens such UV diffusing agents, typical of which is finely divided titanium oxide and zinc oxide.

Other adjunct minor components may also be incorporated into the cosmetic compositions. These ingredients may include coloring agents, opacifiers, and perfumes. Amounts of these other adjunct minor components may range anywhere from 0.001% up to 20% by weight of the composition.

Organic Sunscreens

The inventive cosmetic compositions include an organic sunscreen to provide protection from the harmful effects of excessive exposure to sunlight. Organic sunscreens for purposes of the inventive compositions are organic sunscreen agents having at least one chromophoric group absorbing within the ultraviolet range of from 290 to 400 nm. Chromophoric organic sunscreen agents may be divided into the following categories (with specific examples) including: p-Aminobenzoic acid, its salts and its derivatives (ethyl, isobutyl, glyceryl esters; p-dimethylaminobenzoic acid); Anthranilates (o-aminobenzoates; methyl, menthyl, phenyl, benzyl, phenylethyl, linalyl, terpinyl, and cyclohexenyl esters); Salicylates (octyl, amyl, phenyl, benzyl, menthyl, glyceryl, and dipropyleneglycol esters); Cinnamic acid derivatives (menthyl and benzyl esters, alpha-phenyl cinnamonitrile; butyl cinnamoyl pyruvate); Dihydroxycinnamic acid derivatives (umbelliferone, methylumbelliferone, methylaceto-umbelliferone); Trihydroxycinnamic acid derivatives (esculetin, methylesculetin, daphnetin, and the glucosides, esculin and daphnin); Hydrocarbons (diphenylbutadiene, stilbene); Dibenzalacetone and benzalacetophenone; Naphtholsulfonates (sodium salts of 2-naphthol-3,6-disulfonic and of 2-naphthol-6,8-disulfonic acids); Dihydroxy-naphthoic acid and its salts; o- and p-Hydroxybiphenyldisulfonates; Coumarin derivatives (7-hydroxy, 7-methyl, 3-phenyl); Diazoles (2-acetyl-3-bromoindazole, phenyl benzoxazole, methyl naphthoxazole, various aryl benzothiazoles); Quinine salts (bisulfate, sulfate, chloride, oleate, and tannate); Quinoline derivatives (8-hydroxyquinoline salts, 2-phenylquinoline); Hydroxy- or methoxy-substituted benzophenones; Uric and vilouric acids; Tannic acid and its derivatives (e.g., hexaethylether); (Butyl carbityl) (6-propyl piperonyl)ether; Hydroquinone; Benzophenones (Oxybenzone, Sulisobenzone, Dioxybenzone, Benzoresorcinol, 2,2′, 4,4′-Tetrahydroxybenzophenone, 2,2′-Dihydroxy-4,4′-dimethoxybenzophenone, Octabenzone; 4-lsopropyldibenzoylmethane; Butylmethoxydibenzoylmethane; Etocrylene; and 4-isopropyl-dibenzoylmethane).

Particularly useful are: 2-ethylhexyl p-methoxycinnamate, 4,4′-t-butylmethoxydibenzoylmethane, 2-hydroxy-4-methoxybenzophenone, octyldimethyl p-aminobenzoic acid, digalloyltrioleate, 2,2-dihydroxy4-methoxybenzophenone, ethyl 4-[bis(hydroxypropyl)]aminobenzoate, 2-ethylhexyl-2-cyano-3,3-diphenylacrylate, 2-ethylhexylsalicylate, glyceryl p-aminobenzoate, 3,3,5-trimethylcyclohexylsalicylate, methylanthranilate, p-dimethylaminobenzoic acid or aminobenzoate, 2-ethylhexyl p-dimethylaminobenzoate, 2-phenylbenzimidazole-5-sulfonic acid, 2-(p-dimethylaminophenyl)-5-sulfoniobenzoxazoic acid and mixtures thereof.

Suitable commercially available organic sunscreen agents are those identified under the following table.

TABLE 1

| CTFA Name | Trade Name | Supplier |
|---|---|---|
| Benzophenone-1 | UVINUL 400 | BASF Chemical Co. |
| Benzophenone-2 | UVINUL D-50 | BASF Chemical Co. |
| Benzophenone-3 | UVINUL M-40 | BASF Chemical Co. |
| Benzophenone-4 | UVINUL MS-40 | BASF Chemical Co. |
| Benzophenone-6 | UVINUL D-49 | BASF Chemical Co. |
| Benzophenone-8 | SPECRA-SORB | American Cyanamide |
| | UV-24 | |
| Methoxycinnamate | BERNEL HYDRO | Bernel Chemical |
| Ethyl dihydroxypropyl- | AMERSCREEN P | Amerchol Corp. |
| PABA | | |
| Glyceryl PABA | NIPA G.M.P.A | Nipa Labs. |
| Homosalate | KEMESTER HMS | Hunko Chemical |
| Methyl anthranilate | SUNAROME UVA | Felton Worldwide |
| Octocrylene | UVINUL N-539 | BASF Chemical Co. |
| Octyl dimethyl PABA | AMERSCOL | Amerchol Corp. |
| Octyl methoxycinnamate | PARSOL MCX | Bernel Chemical |
| Octyl salicylate | SUNAROME WMO | Felton Worldwide |
| PABA | PABA | National Starch |
| 2-Phenylbenzimidazole- | EUSOLEX 232 | EM Industries |
| 5-sulphonic acid | | |
| TEA salicylate | SUNAROME W | Felton Worldwide |
| 3-(4-methylbenzylidene)- | EUSOLEX 6300 | EM Industries |
| camphor | | |
| Benzophenone-12 | UVINUL 408 | BASF Chemical Co. |
| 4-Isopropyl | EUSOLEX 8020 | EM Industries |
| dibenzoyl methane | | |
| Butyl methoxy | PARSOL 1789 | Givaudan Corp. |
| dibenzoyl methane | | |
| Etocrylene | UVINUL N-35 | BASF Chemical Co. |

The amount of the organic sunscreens in the cosmetic composition is generally in the range of about 0.01% to about 20%, preferably in the range of about 0.1% to about 10%.

Preferred organic sunscreens are PARSOL MCX and Parsol 1789, due to their effectiveness and commercial availability.

### Use of the Composition

The composition according to the invention is intended primarily as a cosmetic product for topical application to human skin, preferably for cosmetic skin lightening and/or reducing the appearance of oily or greasy skin, more preferably for skin lightening.

In use, a small quantity of the composition, for example about 0.1 to about 5 ml, is applied to exposed areas of the skin, from a suitable container or applicator and, if necessary, it is then spread over and/or rubbed into the skin using the hand or fingers or a suitable device.

### Product Form and Packaging

The cosmetic composition of the invention can be formulated as a lotion having a viscosity (internal fluid friction) of from 4,000 to 10,000 mpas, a fluid cream having a viscosity of from 10,000 to 20,000 mPas or a cream having a viscosity of from 20,000 to 100,000 mPas or above. The composition can be packaged in a suitable container to suit its viscosity and intended use by the consumer. For example, a lotion or fluid cream can be packaged in a bottle or a roll-ball applicator or a propellant-driven aerosol device or a container fitted with a pump suitable for finger operation. When the composition is a cream, it can simply be stored in a non-deformable bottle or squeeze container, such as a tube or a lidded jar.

The invention accordingly also provides a closed container containing a cosmetically acceptable composition as herein defined.

The following specific examples further illustrate the invention, but the invention is not limited thereto.

### EXAMPLES 1-7

A set of compositions within the scope of the present invention were prepared and listed in the Table below. The compositions are in weight per cent.

TABLE 2

| Ingredient Trade and CTFA Name | Phase | 1 | 2 | 3 | 4 | 5 | 6 | 7 |
|---|---|---|---|---|---|---|---|---|
| Stearic acid | A | 14.9 | 14.9 | 12.9 | 17.9 | 14.0 | 14.0 | 14.0 |
| Sodium cetearyl sulfate | A | 1.0 | 1.0 | 1.5 | 1.5 | 1 | 1 | 1 |
| Myrj 59 | A | 2.0 | 1.5 | 2 | 2 | 2 | 2 | 2 |
| Span 60 | A | 2.0 | 1.5 | 2 | 2 | 2 | 2 | 2 |
| Propyl paraben | A | 0.10 | 0.10 | 0.10 | 0.10 | 0.10 | 0.10 | 0.10 |
| BHT | A | 0.1 | 0.1 | 0.1 | 0.1 | 0.1 | 0.1 | 0.1 |
| Dimethicone | A | | 0.50 | 0.75 | | 0.75 | 0.75 | 0.75 |
| Water | B | BAL* | BAL | BAL | BAL | BAL | BAL | BAL |
| EDTA | B | 0.04 | 0.04 | 0.04 | 0.04 | 0.04 | 0.04 | 0.04 |
| Pemulen TR 2 | B | | 0.10 | 0.05 | | 0.05 | 0.05 | 0.05 |
| Methyl paraben | B | 0.15 | 0.15 | 0.15 | 0.15 | 0.15 | 0.15 | 0.15 |
| Parsol MCX (organic sunscreen) | C | 0.75 | 1.25 | 1 | 1 | 0.75 | 0.75 | 0.75 |
| Parsol 1789 (organic sunscreen) | C | 0.40 | | 0.4 | 0.4 | 0.4 | 0.4 | 0.4 |
| Micronized Titanium oxide | C | | | | | 0.2 | 0.2 | 0.2 |
| Propylene glycol | D | | | | | 8 | 8 | 8 |
| Transcutol | D | | | | | 4 | 4 | 4 |
| 4-cyclopentyl methyl resorcinol | D | 0.05 | 2.0 | 2.0 | 3.5 | | | |
| 4-cyclohexyl methyl resorcinol | D | | | | | 2.5 | | |
| 4-cyclothiane methyl resorcinol | D | | | | | | 3.51 | |
| 4-cycloamido methyl resorcinol | D | | | | | | | 5.0 |

*BAL = balanced to 100%

The compositions of Examples 1-7 in the Table above were prepared in the following fashion. Phase A is heated at 75° C. Phase B is heated to 75° C. in a container separate from that of Phase A. Thereafter the phases are combined with mixing with heat being turned off. Phase C was premixed and warmed then added immediately after phase A and B mixed. Phase D is pre-dissolved and added into the main pot at 60° C. The mixture is cooled until 40° C. and then packed.

### EXAMPLE 8

This example demonstrates the skin lightening activity of 4-cyclo-hexyl methyl resorcinol.

### Mushroom Tyrosinase Assay

Mushroom tyrosinase inhibition is indicative of reduction in melanin synthesis, thereby showing skin lightening effect.

Reagents:

Assay buffer: phosphate (100 mM, pH 7.0)
Mushroom tyrosinase stock solution (Sigma-Aldrich, Batch # 023K7024): 0.2 mg/ml in assay buffer
L-DOPA stock solution: 1.05 mM in assay buffer
Test compound stock solutions: 10 mM in DMSO
   Assay Conditions:
Mushroom tyrosinase: 0.1 mg/ml in assay buffer
L-DOPA: 0.5 mM in assay buffer
Test compounds: various concentrations, 2.5% final [DMSO]
Temperature: room temperature
   Procedure:

Test compounds (5 uL of stock solutions) are added into wells of a 96-well plate, followed by L-DOPA (L-3, 4-Dihydroxyphenylalanine; 95 uL of stock solution). The reaction is started by adding mushroom tyrosinase (100 uL of stock solution) into each well and the absorbance is monitored at 490 nm over a time period of 30 sec to 2 min. The initial reaction velocity in the presence or absence of test compounds is calculated (Δ490 nm/ min) and the % inhibition of test compounds is calculated using the following equation:

$$\% Inhibition = ( 1 - \frac{{v}_{i} - v}{{v}_{0} - v} ) * 100$$

where v₀ is the initial reaction velocity in the absence of compound, v is the slope of the reaction in the absence of mushroom tyrosinase and vᵢ is the slope of the reaction in the presence of test compound. The data (% inhibition vs. [test compound]) is fitted using data analysis software and the concentration of test compound at 50% inhibition (IC50) is determined from the fitted data.

TABLE 3

| Name | IC50 (uM) |
|---|---|
| 4-cyclohexylmethyl resorcinol (CHMR) | 0.95 |
| 4-ethyl resorcinol (ER) | 1.10 |

The IC50 value refers to the skin lightener concentration that results in 50% tyrosinase inhibition relative to the control (with a goal being obtaining maximum tyrosinase inhibition at minimum concentration).

The data appear to show that cyclohexyl methyl resorcinol and ethyl resorcinol have comparable potency.

### EXAMPLE 9

This example demonstrates the skin sebum suppressive activity of 4-cyclo-hexyl methyl resorcinol.

Sebocyte Assay Procedure:

Secondary cultures of human sebocytes obtained from an adult male were grown in 96-well tissue culture plates (Packard) until three days post-confluence. Sebocyte growth medium consisted of Clonetics Keratinocyte Basal Medium (KBM) supplemented with 14 ug/ml bovine pituitary extract, 0.4 ug/ml hydrocortisone, 5 ug/ml insulin, 10 ng/ml epidermal growth factor, 1.2×10⁻¹⁰ M cholera toxin, 100 units/ml penicillin, and 100 ug/ml streptomycin. All cultures were incubated at 37° C. in the presence of 7.5% CO₂. Medium was changed three times per week.

On the day of experimentation, the growth medium was removed and the sebocytes washed three times with sterile Dulbecco's Modified Eagle Medium (DMEM; phenol red free). Fresh DMEM was added to each sample (triplicates) with 2-microliters of test agent solubilized in ethanol. Controls consisted of addition of ethanol alone. Each plate was returned to the incubator for 20-hours followed by the addition of ¹⁴C-acetate buffer (5 mM final concentration, 56 mCi/mmol specific activity). Sebocytes were returned to the incubator for 4-hours, after which, each culture was rinsed 3-times with phosphate buffered saline to remove unbound label. Radioactive label remaining in the sebocytes was determined using a Packard TopCount scintillation counter. Statistical significance (p value) was calculated using student's t-test. The results that were obtained are summarized in Table 1. Phenol Red, a known sebum suppressive agent, was employed as a positive control.

TABLE 4

| Treatment | % of Control (ethanol) | p-value |
|---|---|---|
| 10 uM CHMR | 98.9 | 0.3657 |
| 100 uM CHMR | 69.3 | 0.0036 |
| 28 uM Phenol Red | 84.4 | 0.0581 |
| 280 uM Phenol Red | 45.9 | 0.0007 |

The data appear to show that, at a concentration on the order of about 100 micro-molar, CHMR (cyclohexyl methyl resorcinol) provides a statistically significant reduction in sebocyte lipid synthesis as compared to the vehicle control.

It should be understood that the specific forms of the invention herein illustrated and described are intended to be representative only. Changes, including but not limited to those suggested in this specification, may be made in the illustrated embodiments without departing from the clear teachings of the disclosure. Accordingly, reference should be made to the following appended claims in determining the full scope of the invention.

What is claimed is:

1. A compound of general formula I: 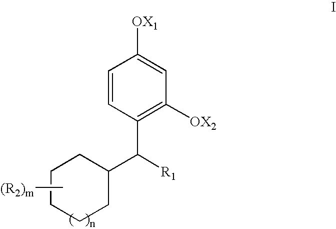
Wherein
X₁ and/or X₂ represents hydrogen (H), linear or branched, saturated or unsaturated C₁-C₁₂ alkyl, alkenyl, or acyl groups;
R₁ and/or R₂ represents hydrogen (H), linear or branched, cyclic or acyclic, saturated or unsaturated C₁-C₁₂ alkyl, alkenyl, cycloalkyl, or cycloalkenyl group;
n=0,1; and
when n=0, the ring is cyclopentyl with or without one heteroatom from O, N or S and/or with or without one double bond; or
when n=1, the ring is cyclohexyl with or without one heteroatom from O, N or S and/or with or without one double bond; and
m is an integer between 1 and 6.

2. A cosmetic method of reducing the appearance of oily or greasy skin comprising applying to the skin a compound according to claim 1.

3. A cosmetic method of skin lightening comprising applying to the skin a compound according to claim 1.

4. A compound of general formula B: 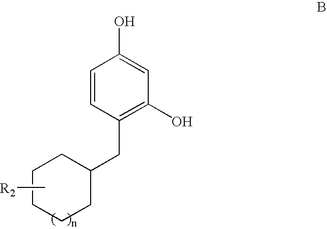
Wherein
R₂=hydrogen (H), linear or branched, cyclic or acyclic, saturated or unsaturated C₁-C₁₂ alkyl, alkenyl, cycloalkyl, or cycloalkenyl group;
n=0, 1; and
when n=0, the ring is cyclopentyl with or without one heteroatom from O, N or S and/or with or without one double bond; or
when n=1, the ring is cyclohexyl with or without one heteroatom from O, N or S and/or with or without one double bond.

5. A cosmetic method of skin lightening and/or reducing the appearance of oily or greasy skin comprising applying to the skin a compound of general formula B according to claim 4.

6. A cosmetic composition comprising:
(a) about 0.0001 wt. % to about 50 wt. % of a compound of general formula I according to claim 1; and
(b) a cosmetically acceptable carrier.

7. The composition of claim 6, further comprising an organic sunscreen selected from the group consisting of Benzophenone-1, Benzophenone-2, Benzophenone-3, Benzophenone-4, Benzophenone-8, DEA, Methoxycinnamate, Ethyl dihydroxypropyl-PABA, Glyceryl PABA, Homosalate, Methyl anthranilate, Octocrylene, Octyl dimethyl PABA, Octyl methoxycinnamate (PARSOL MCX ), Octyl salicylate, PABA, 2-Phenylbenzimidazole-5-sulphonic acid, TEA salicylate, 3-(4-methylbenzylidene)-camphor, Benzophenone-6, Benzophenone-12, 4-Isopropyl dibenzoyl methane, Butyl methoxy dibenzoyl methane (PARSOL 1789), Etocrylene, and mixtures thereof.

8. The composition of claim 6, wherein said compound of general formula I is present in an amount of about 0.01 wt. % to about 5 wt. %.

9. The composition of claim 1, wherein said compound of general formula I is selected from the group consisting of 4-cyclopentyl methyl resorcinols, 4-cyclohexyl methyl resorcinols, and mixtures thereof.

10. The cosmetic composition of claim 6, further comprising a skin benefit agent selected from the group consisting of alpha-hydroxy acids and esters, beta-hydroxy acids and esters, polyhydroxy acids and esters, kojic acid and esters, ferulic acid and ferulate derivatives, vanillic acid and esters, dioic acids and esters, retinol, retinal, retinyl esters, creatine, hydroquinone, t-butyl hydroquinone, mulberry extract, licorice extract, resorcinol derivatives, and mixtures thereof.

11. The cosmetic composition of claim 7, wherein said organic sunscreen is present in an amount of about 1 wt % to about 10 wt % of said cosmetic composition; and
wherein the weight ratio of said organic sunscreen to said compound of general formula I is about 10000:1 to about 1:10000.

12. The cosmetic composition according to claim 10 wherein said skin benefit agent is selected from the group consisting of alpha-hydroxy acids, beta-hydroxy acids, polyhydroxy acids, hydroquinone, t-butyl hydroquinone, 4-substituted resorcinol derivatives, and mixtures thereof.

13. A cosmetic method of skin lightening comprising applying to skin the composition of claim 6.

14. A cosmetic method of reducing the appearance of oily or greasy skin comprising applying to skin the composition of claim 6.

15. A process for synthesizing a compound of general formula I, comprising: 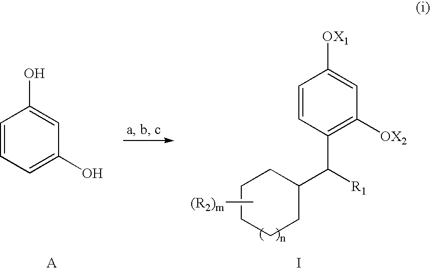
Wherein a, b, c represent reaction steps, reagents and conditions:
(a) alpha-alkylbenzyl alcohol derivative or substituted alpha-alkylbenzyl alcohol derivative, acid catalyst;
(b) hydrogen, catalyst, solvent;
(c) acylation or alkylation; and
wherein
R₁ and/or R₂ represents hydrogen (H), linear or branched, cyclic or acyclic, saturated or unsaturated C₁-C₁₂ alkyl, alkenyl, cycloalkyl, or cycloalkenyl group;
n=0, 1; and
when n=0, the ring is cyclopentyl with or without one heteroatom from O, N or S and/or with or without one double bond; or
when n=1, the ring is cyclohexyl with or without one heteroatom from O, N or S and/or with or without one double bond; and
m is an integer between 1 and 6.

16. A process for synthesizing a compound of general formula I, comprising: 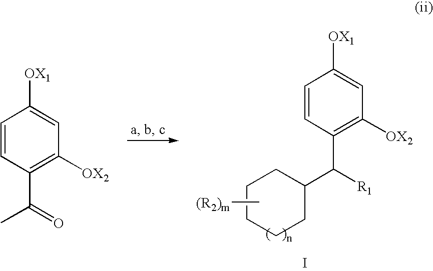
wherein a, b, c represent reaction steps, reagents and conditions:
(a) cycloalkylmagnesium halide derivative or substituted cycloalkylmagnesium halide derivative, organic solvent;
(b) acidic conditions;
(c) hydrogen, catalyst, solvent; and
wherein
R₁ and/or R₂ represents hydrogen (H), linear or branched, cyclic or acyclic, saturated or unsaturated C₁-C₁₂ alkyl, alkenyl, cycloalkyl, or cycloalkenyl group;
n=0, 1; and
when n=0, the ring is cyclopentyl with or without one heteroatom from O, N or S and/or with or without one double bond; or
when n=1, the ring is cyclohexyl with or without one heteroatom from O, N or S and/or with or without one double bond; and
m is an integer between 1 and 6.
